(19) United States
(12) Patent Application Publication
(10) Pub. No.: US 2003/0031652 A1
(43) Pub. Date: Feb. 13, 2003

(54) Systems and methods for inducing mixed chimerism

(76) Inventors: Bernhard Hering, Minnetonka, MN (US); Zhiguang Guo, White Bear Lake, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 09/855,027

(22) Filed: May 14, 2001

Related U.S. Application Data
(60) Provisional application No. 60/284,005, filed on Apr. 16, 2001.

(51) Int. Cl. A61K 48/00; C12N 5/08

(52) U.S. Cl. 424/93.21; 424/93.7; 435/372

(57) ABSTRACT

A mixed chimeric immune system is created for a variety of treatments and techniques. Mixed chimerism is established in a patient without significant risk of graft-versus-host-disease (GVHD) by administering a cell transplant from a donor to a recipient along with a conditioning treatment and an immune blockade treatment.

### FIELD OF THE INVENTION

The invention relates to inducing tolerance to transplanted materials such as allogeneic, xenogeneic, and autogeneic materials transplanted into a patient and to restoring self-tolerance in the case of autoimmunity conditions. More specifically, the invention relates to creating mixed chimerism in patients and treating graft rejection, malignant cell growth, and autoimmune conditions.

### BACKGROUND OF THE INVENTION

Organ transplantation has saved many lives and greatly improved the quality of life for organ recipients; however, the recipients must be treated for the rest of their lives with powerful drugs that suppress their immune system. Unfortunately, these immunosuppressant drugs make the recipient vulnerable to disease and block the body's natural cancer resistance. While the immunosuppressant drugs are designed to prevent rejection of the transplanted organ, these drugs are not always effective and transplanted organs are often rejected after a short time (acute rejection) or over the long term (chronic rejection). For instance, only about 50% of heart, lung, or liver transplants that function after one year are still functioning at ten years.

The ability for a patient to successfully tolerate transplanted organs is referred to as tolerance. Just as the human body's immune system normally tolerates its own organs, a condition called self-tolerance, an organ recipient would ideally tolerate a donated organ without the need for long-term immunosuppressant drugs. Tolerance without the need for continued use of such immunosuppressant drugs is one of the principle goals of the field of transplantation. While many attempts are being made to achieve this goal, our understanding of the immune system is still incomplete and no approach has yet to reach this goal in a manner suitable for a clinical setting.

T-cells are the immune system cells that are chiefly responsible for transplant rejection and autoimmune disorders. One approach to achieving tolerance has been to destroy a recipient's bone marrow cells, which produce the T-cells, and completely replace them with a donor's bone marrow. The destruction of bone marrow is termed myeloablation. Since bone marrow plays a key role in the immune system, the recipient begins to use the “donated” immune system. The complete myeloablation and replacement of bone marrow causes the recipient to use only the donated immune system, a condition termed full chimerism. The major obstacle to successful bone marrow transplantation is the toxicity associated with myeloablation and graft-versus-host disease (GVHD). Myeloablation weakens the immune system and makes a patient vulnerable to infections. GVHD is a common complication of allogeneic bone marrow transplants (i.e., bone marrow transplants from a donor other than an identical twin). GVHD is a condition where the donor's bone marrow, especially its T-cells, attack the patient's own organs and tissue, including the skin, liver, and gastrointestinal tract. A severe case of GVHD is often fatal.

Another approach to creating tolerance has been to use agents to directly block the T-cell response to the transplanted organ. The T-cell response includes the interaction of molecules on the surface of the T-cells with molecules on other cells. The T-cells have certain molecules, (e.g., CD154 and CD28) that interact with receptor molecules in other cells (e.g., the CD40 receptor and the B7 receptor molecules, respectively). Drugs that block these interactions (anti-CD154 antibody, which blocks the CD154-to-CD40 receptor interaction and CTLA4Ig, which interferes with CD28-to-B7 interaction) can interfere with the organ rejection process. While high levels of anti-CD 154 antibody have been reported to block GVHD, the level of these drugs necessary to completely interfere with the organ rejection process can create problems similar to conventional immunosuppressant drugs.

Recently, it has been suggested that tolerance might be achieved as a result of successfully inducing a condition termed mixed chimerism. In mixed chimerism, the recipient would use both their original immune system and a donated immune system. The donor and recipient immune systems would co-exist and cooperate in the recipient. In addition to potentially creating tolerance for transplants, the ability to successfully establish mixed chimerism could be used as a therapy for autoimmune diseases. Part of the challenge of creating mixed chimerism, however, is that the donor and recipient T-cells initiate immune systems attack each other or the recipient, which can result in GVHD. Although mixed chimerism should reduce the risks of GVHD compared to full chimerism, scientists have yet to discover how to consistently and safely establish mixed chimerism without generating GVHD.

Several approaches for establishing mixed chimerism have been attempted. In general, these approaches use techniques that severely suppress the functions of the recipient's bone marrow and/or immune system for a prolonged period of time as part of the treatment. Such severe and lengthy suppression has been thought necessary to let donor and recipient T-cells adapt to a state of coexistence. Suppression of bone marrow and immune functions is typically achieved with irradiation therapy and/or high doses of drugs such as fludarabine phosphate, cyclophosphamide, and busulfan. An important measure of severe suppression is whether the patient exhibits neutropenia, a condition indicating a shortage of neutrophils (white blood cells that digest and destroy particles and fight infections).

Suppression of the immune system, however, is undesirable because it leaves patients

vulnerable to opportunistic infections and disease during the course of such treatments. As a result, the rate of complications and the cost of treatment are increased. Suppression of the bone marrow not only suppresses the immune system but also suppresses the body's ability to make blood (termed hematopoiesis). Damage to the blood-making ability severely impacts the recipient's health.

Removal of T-cells from donor marrow is another typical step that has been attempted in an effort to help prevent GVHD. The concept behind this step is that removing most of the donor T-cells will decrease the risk of an attack on the recipient by the donor immune system.

Removal of T-cells, however, is a labor-intensive process that increases the risks for infection and causes the loss of stem cells and facilitating cells that the donated bone marrow needs to be able to survive in its new host.

Some experimental organ transplantation treatments have attempted a two step process in patients with myeloma. The process involved inducing bone marrow transplantation from a living donor to establish chimerism and then following with transplant of the organ several weeks later; unfortunately, this process had a high risk of damage to the transplanted organ. Further, persons that are waiting for organ transplants are usually very ill, so the time between organ transplantation can be crucial. The extra time increases medical complications and cost.

Despite past attempts to achieve mixed chimerism, no consistent and safe approach has been developed for establishing mixed chimerism in a patient without significant risk of generating GVHD. For instance, approaches that deplete donor T cells from the bone marrow inoculum prior to bone marrow transplantation were intended to reduce the risk of GVHD but have also reduced the chances of successful bone marrow transplantation. These past attempts severely suppressed the bone marrow and/or immune system and caused neutropenia.

The ability to successfully establish mixed chimerism without significant risk of generating GVHD would be a major step in organ transplantation, the treatment of autoimmune diseases, cancer treatments, and pathological conditions such as hemoglobinopathies. The ability to not only reduce GVHD but also have only a small suppressive effect on bone marrow functions and immune system functions, to avoid neutropenia, and to avoid T-cell depletion steps would be another major step. The further ability to transplant bone marrow and follow with an organ or cell transplant in only a few days would represent another major step. A simultaneous bone marrow and organ transplant would be yet another major step.

### SUMMARY OF THE INVENTION

The present invention presents effective techniques and treatments for producing mixed chimerism without significant risk of generating GVHD. These techniques have only a small suppressive effect on the immune system and bone marrow functions and cause little or no neutropenia compared to other techniques. No step to treat extracted donor bone marrow to deplete T-cells is required. The techniques make it possible to introduce bone marrow and a transplanted organ or tissue within a few days of each other and, in some cases, on the same day, thereby making feasible the transplantation of organs and tissue from a non-living donor.

The techniques use the synergistic effects of a combination of reduced levels of pre-transplant immune suppression coupled with lower levels of post-transplant immune blockade. Because the techniques are generally mild in their suppression of a patient's bone marrow activity, the trauma to a patient's blood supply and immune system is minimized and the patient is able to adapt more rapidly to the infusion of donor bone marrow. Since the patient is less traumatized by the pre-treatment regimen, it is possible to decrease the amount and timing of post-transplant immune blockade therapy required to prevent GVHD. The present invention recognizes the unexpected result that these two effects actually enhance each other and are, therefore, synergistic with each other. By recognizing the synergistic effects of a combination of reduced levels of pre-transplant immune suppression coupled with lower levels of post-transplant immune blockade, the techniques of the present invention provide for treatments that rapidly induce mixed chimerism with minimal immune and hematopoietic suppression without inducing GVHD.

One treatment in accordance with a preferred embodiment of the present invention involves a conditioning step of administering fludarabine phosphate and/or cyclophosphamide prior to infusing donor bone marrow cells and blocking T-cell activity after bone marrow infusion by using agents that block or interfere with CD40 receptor/CD 154 (called CD40 ligand), and CD28/B7 receptors. T-cell activity may also be blocked by Rapamycin or a comparable equivalent. MR1, 5C8, and IDEC-131 are antibody agents for blocking CD40L ligand-to-CD40 receptor interaction and CTLA4Ig is an agent that interferes with CD28-B7 receptor interaction. Since effective blocking of T-cell activity prevents GVHD, the harsh suppression of the recipient immune system and/or bone marrow cell activity that is generally favored in conventional treatments is simply not needed. Instead, only a much less toxic conditioning regimen of agents such as Busulfan, fludarabine phosphate and/or cyclophosphamide is required. Because a harsh treatment of the immune system is unnecessary, mixed chimerism can be achieved more rapidly and with only a mild regimen of immune suppression. Since mixed chimerism is rapidly established, the risks of complications and unfavorable reactions are minimized.

One advantage of the techniques of the present invention is that they require only a brief inhibition of the immune function. In contrast, existing techniques for inducing mixed chimerism require a lengthy suppression of immune functions. As a result, the patient is at a much greater risk of succumbing to opportunistic maladies and must be maintained in an uncomfortable and costly hospital environment. Because the immune system is mildly inhibited by the techniques of the present invention as compared to conventional treatments, the result is that the patient's immune system recovers to normal levels more quickly and the onset of mixed chimerism is accelerated.

An advantage of the invention is that the techniques, in contrast to typical conventional techniques, do not require that donor bone marrow extracted from a donor be depleted of its T-cells. As a result, recovery and onset of mixed chimerism is accelerated. The elimination of the T-cell depletion step saves time, money, and increases reproducible and consistent results.

The techniques of the present invention also enable transplantation of organs and tissue with much less matching than conventionally practiced transplantation protocols. Mismatched donors and recipients may be used without the elaborate matching process that is conventionally required. The invention facilitates a higher degree of mismatching between donor and recipient that was previously possible and extends bone marrow and stem cell transplants to haploidentical and even completely mismatched donor-recipient pairs, including transplants from cadaveric bone marrow and peripheral blood stem cell donors.

Another advantage of the invention is that mixed chimerism establishes the graft-versus-tumor effect (GVT). The beneficial effects of GVT are difficult to separate from the detrimental effects of GVHD but these techniques prevent GVHD and promote mixed chimerism such that GVT may be achieved. Inducing GVT in a cancer patient causes their body to attack the cancer. Inducing GVT by the techniques of the present invention is a treatment for cancers.

The course of treatments may optionally include use of agents like anti-lymphocyte serum (ALS) and/or infusion of donor cells, for example spleen cells or blood cells, prior to bone marrow cell transplantation. This infusion generally enhances the establishment of mixed chimerism but is not necessary.

The techniques and treatments of the invention are applicable not only to organ transplant but also to cell transplants, treating autoimmune diseases, preventing autoimmunity and related diseases in at-risk patients and, treating cancer and other pathological conditions such as hemoglobinopathies. Indeed, this invention enables an organ transplant and bone marrow transplant to be performed simultaneously or on the same day.

### BRIEF DESCRIPTION OF THE DRAWINGS

FIG. 1 is an illustration of treatments for inducing mixed chimerism.

FIG. 2 is an illustration that compares the invention's impact on the immune system to prior art treatments.

FIG. 3 is an illustration of treatments for inducing mixed chimerism that include ALS.

FIG. 4 is an illustration of treatments for inducing mixed chimerism that include donor cell pretreatment.

FIG. 5 is an illustration of treatments for inducing mixed chimerism and transplanting tissue.

FIG. 6 is an illustration of treatments for transplanting tissue and bone marrow within 24 hours.

FIG. 7 shows how a preconditioning treatment of FL and CY reduces lymphocytes in the peripheral blood of C57BL/6 mice without reducing granulocyte and/or neutrophil populations.

FIGS. 8A and 8B show lymphocytes (R1) in mice given FL and CY conditioning treatments.

FIGS. 9A and 9B show control mice lymphocytes in the experiment of FIG. 8.

FIG. 10 shows deletion of Vβ5+ and Vβ11+ peripheral CD4+ cells in chimeric C57BL/6 Mice (at 20 Weeks Post-BMT).

FIG. 11 compares the donor specific cytokine secreting T-cells in chimeric NOD mice compared to NOD mice without Chimerism.

FIG. 12 compares PHA mitogen specific cytokine secreting T cells in chimeric and non-chimeric NOD mice.

FIG. 13 compares the onset of diabetes in chimeric and non-chimeric NOD mice.

FIG. 14 compares the survival of transplanted islets in chimeric and non-chimeric mice.

FIG. 15 shows blood glucose levels in diabetic NOD mice after simultaneous islet and bone marrow transplantation with ALS treatment, preconditioning with FL and CY, and immune blockade with Rapamycin.

FIG. 16 shows donor chimerism levels in the hematopoietic organs of mixed chimers at 20 weeks post-bone marrow transplant.

### DETAILED DESCRIPTION OF THE PREFERRED EMBODIMENT

The Immune System

A person's own immune system normally does not attack the person, a condition called self-tolerance. The immune system also has the ability to identify and respond to invading or foreign agents, an ability generally termed acquired immunity. Acquired immunity uses two main mechanisms: B-cell immunity (also termed humoral immunity) and T-cell immunity (also termed cell-mediated immunity). B-cell immunity is mediated by B-cells and involves the creation of antibodies. T-cell immunity is mediated by T-cells and involves the activation of lymphocytes that kill the foreign agents. Both T-cells and B-cells are termed lymphocytes. Both B-lymphocytes (B-cells) and T-lymphocytes (T-cells) respond when they recognize molecular-sized targets, which are called antigens. Lymphocytes have distinctive molecules on their surface that allows them to be distinguished from other cells. Once the B-cells or T-cells respond to an antigen, they begin to proliferate and send out chemical signals that cause an amplification, or cascade, of events that activate many cells and eventually causes the destruction of the foreign cells that bear the offending antigen.

There are three major groups of T-cells: two types of regulatory T-cells, termed Helper T-cells and Suppressor T-cells, and the Cytotoxic T-cells. Regulatory T-cells are helper cells that help to activate other cells in the immune system. Cytotoxic T-cells directly attack cells that have been infected by viruses or transformed by cancer and are chiefly responsible for the rejection of tissue and organ grafts. T-cells work by secreting cytokines or, more specifically, lymphokines. Lymphokines (also secreted by B cells) are chemical messengers that evoke many reactions from various cells. A single cytokine may have many functions and several cytokines may be able to produce the same effect. Many cytokines have initial names but, as their basic structure is identified, they are renamed as “interleukins” and are denoted as IL-1, IL-2, and so forth.

GVHD is thought to be mediated by T-cells in several ways. T-cells are generally active in the T-cell immunity system, so generally suppressing their functions or destroying them can counteract GVHD. Suppressing CD8-positive T-cells is an example of this approach. Another way that T-cells contribute to GVHD is by their CD40 ligand (also called CD154) on their surface binding to the CD40 receptor on dendritic or macrophage cells; since these cells “present” the antigens that are on foreign tissue, blockage of this interaction helps to prevent the T-cell immune system from attacking the foreign tissue. Another GVHD T-cell mediation mechanism involves the T-cell's CD28 ligand binding the B7 receptor (i.e., receptors termed CD80 (B7-1) or CD86 (B7-2)) on antigen-presenting cells (APCs) such as dendritic cells.

Agents for Controlling the Immune System

There is a class of drugs termed myelosuppressants that inhibit bone marrow cell function. The function of bone marrow cells includes making T-cells and hematopoiesis, which means making cells and materials required for blood to function. So generally inhibiting bone marrow cell function inhibits the function of the immune system and inhibits hematopoiesis. Another class of drugs termed immunosuppressants are more directly targeted to blocking only the immune system, for example by interfering with an important T-cell immunity receptor. Some of these immunosuppressant drugs are chemotherapy agents, which include alkaloids, alkylating agents, antimetabolites, enzymes, hormones, platinum compounds, and new drugs.

Alkylating agents are toxic chemicals that tend to react with DNA with the result that they destroy the DNA or cause it to be come crosslinked. They tend to preferentially kill proliferating cells, especially bone marrow cells and are generally myelosuppressants (inhibitors of bone marrow cell activities). Most alkylating agents can be classified as nitrogen mustards or nitrosoureas. Nitrogen mustards include mechlorethamine and chlorambucil, and melphalan; but the most commonly used alkylating agent is cyclophosphamide. It can be given in a variety of ways and dosages unlike many of the other nitrogen mustards. Ifosphamide is an alkylating agent closely related to cyclophosphamide. Nitrosoureas include carmustine, lomustine and semustine. Other alkylating agents include cyclophosphamide, busulfan, dacarbazine, hydroxymethylmelamine, thiotepa and mitocycin C.

FLUDARA is a trade name for fludarabine phosphate. Fludarabine phosphate is changed in the body to a metabolite that appears to act by inhibiting DNA polymerase alpha, ribonucleotide reductase and DNA primase, thus inhibiting DNA synthesis. It acts on a very wide range of cell types and generally stops or slows the multiplication of all cells. It is a myelosuppressant but at properly controlled levels is not myeloablative.

Cyclosporine (CSA) is an immunosuppressant that blocks gene transcription of IL-2 and other lymphokines so that T-cells do not proliferate and the immune response to a foreign antigen is suppressed. Its primary target is helper T lymphocytes, with little effect on other aspects of the immune response. CSA and tacrolimus are thought to bind to immunophilin. The CSA-immunophilin complex in turn binds to and blocks a phosphatase called calcineurin, which is needed to activate enhancers/promoters of certain genes, including those for transcription of IL-2 (and other early activation factors).

RAPAMUNE is a trade name for Sirolimus, also known as rapamycin, an immunosuppressant. Sirolimus has been shown to block T-cell activation and proliferation by blocking the response of T and B cells to cytokines, thereby preventing cell cycle progression at stage G1 and consequently blocking T-cell and B-cell proliferation. More specifically, sirolimus blocks T lymphocyte proliferation in response to IL-2 and blocks the stimulation caused by ligand binding of the T-cell's CD28 molecule. It is thought to do this by blocking activation of the kinase referred to as mammalian target of rapamycin or “mTOR”, a serine-threonine kinase that is important for cell cycle progression. It generally has synergy with cyclosporine (CSA) in vitro as well as in animal and clinical studies. It is soluble in dimethylsulfoxide (DMSO) and methanol.

Cyclophosphamide (CY) is an alkylating agent that may be used as an immune suppressant. It generally suppresses the B-cell immunity system and the T-cell immunity system by acting generally against proliferating cells. It has trade names such as CYTOXAN. As an immunosuppressant its most important effect in controlling GVT and GVHD is thought to be clonal destruction. T-cells and B-cells normally will proliferate in response to a foreign antigen so that there are many of them that respond to the same antigen; the proliferation is a key part of the immune system's amplification process. The proliferating cells are especially vulnerable to CY so that CY tends to kill all of these proliferating cells and thereby stop the amplification of the initial response to the foreign antigen. At properly controlled levels CY is not myeloablative.

Busulfan, also called Myelosan or Busulphan, is an alkylating agent that is a myelosuppressant. It has trade names such as BUSULFEX, or MYELERAN. Like other alkylating agents, it generally is believed to cross-link the DNA of proliferating cells so they die.

T-cells express a surface molecule called the CD40 ligand that binds the CD40 receptor on dendritic cells. The CD40 ligand-to-CD40 receptor binding event is important for activating T-cells to recognize a foreign antigen and for amplifying the immune response. MR1 is an agent that interferes with this binding event in mice. MR1 is an antibody against the CD40 ligand, i.e., the “antibody recognizes” or “the antibody binds” it. Other antibodies exist that also bind to the CD40 ligand or receptor in other species, for example the antibodies 5C8 and IDEC-131 that bind the CD40 ligand in humans.

Another GVHD T-cell mediation mechanism involves the T-cell's CD28 ligand binding to the B7 receptor (i.e., receptors termed CD80 (B7-1) or CD86 (B7-2)) on antigen-presenting cells (APCs) such as dendritic cells. This binding event amplifies the response of the immune system to a foreign antigen. The molecule CTLA4 (also called CD 152) binds the B7 receptor so that there is not a CD28-to-B7 binding event. CTLA4 is a natural “off switch” that is present at very low concentrations in the body. REPLIGEN, Inc., manufactures CTLA4-Ig which is modeled after CTLA4 and also acts as an “off switch” by competitively inhibiting the binding of B7 to CD28. CTLA4-Ig and LEA29Y, a mutant form of CTLA4-Ig counteracts GVHD.

Tacrolimus, also called PROGRAF or FK506, is many times more potent than cyclosporine. The critical difference is that it inhibits interleukin 2 expression and synthesis, and has a specific action on T-helper lymphocytes.

Anti-lymphocyte globulin (ALG) is a mixture of antibodies against lymphocytes and acts as a general immunosuppressant. Anti-thymocyte globulin (ATG) acts in a similar fashion to ALG and is generally its equivalent. Antilymphocyte serum (ALS) is a serum of polyclonal antibodies against lymphocytes and acts in a similar fashion to ALG and is generally its equivalent.

Medical professionals and scientists use the term myeloablative in a variety of ways. Myeloablative literally means to kill bone marrow cells, but the word is often used to describe only procedures that kill most or all of a patient's bone marrow cells. The methods described herein are nonmyeloablative in the sense that they do not kill all or most of a patient's bone marrow. These methods are mildly myeloablative in the sense that they cause the death of only a small percentage of a patient's bone marrow cells.

Neutropenia

The term neutropenia is also used in different ways. Neutropenia means a decline in the number of neutrophils, for instance in the blood or liver (Dorland's Medical Dictionary, 28th Ed.). The term neutropenia, however, can also mean a marked decline or shortage of neutrophils. The invention may cause a small decrease in neutrophils but the invention avoids neutropenia in the sense that it does not cause a marked decline or shortage of neutrophils.

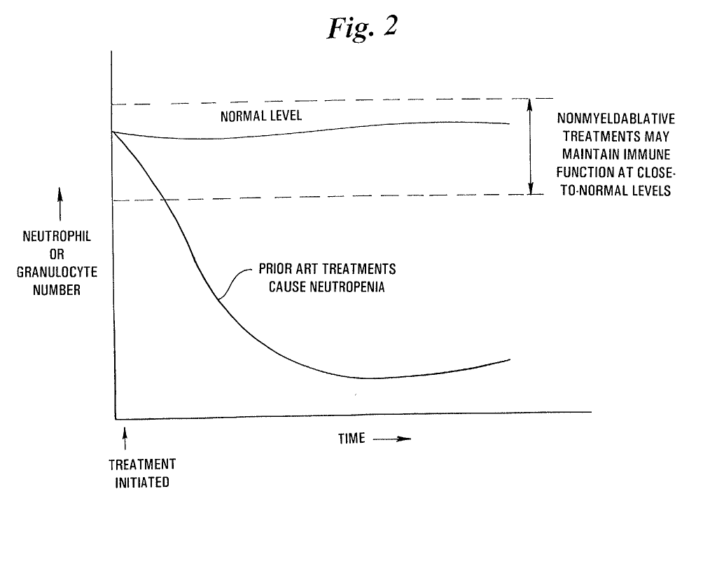

Neutrophils are a type of granulocyte, which is a white blood cell. Lymphocytes are also white blood cells. These cell types are involved in immune function. In contrast to conventional treatments, the conditioning treatment of the invention reduces the number of lymphocytes in the patient's blood but has a small impact on the number of granulocytes or neutrophils. The conditioning treatment is specifically directed to lymphocytes in the sense that it markedly and transiently decreases lymphocyte numbers (thus causing a drop on the total white blood cell count) without markedly decreasing neutrophil and/or granulocyte counts (FIGS. 2 and 7).

A measurement of the number or change in number of neutrophils or granulocytes is sufficient to indicate if a patient is suffering from neutropenia. A related condition is granulocytopenia, a condition indicated by a marked decrease in granulocytes and certain symptoms (Dorland's Medical Dictionary, 28th Ed.).

Graft Versus Host Disease (GVHD)

Current science leaves open the question of whether or not graft-versus-tumor (GVT) effects can be induced in the absence of clinically overt GVHD. Current methods that tend to promote GVT tend to also promote GVHD but suppressing GVHD tends to also suppress GVT. GVHD occurs in an early form termed acute GVHD that occurs within about the first three months following an allogeneic bone marrow cell transplant and a late form termed chronic GVHD. Acute GVHD is currently believed to be caused chiefly by the T-lymphocytes that are part of the transplanted bone marrow cell. The T-lymphocytes attack the patient's skin, liver, stomach, and/or intestines.

One approach to preventing GVHD is T-cell depletion (e.g., elutriation, monoclonal antibody treatment, and use of columns). In this approach the donor bone marrow cells are subjected to a time consuming and labor-intensive process to remove T-cells, for instance by column chromatography or separation by size and density. Removal of too many of these cells, however, will negatively impact the engraftment of donor stem cells and may prevent GVT. GVT is desired when cancer is present because it will attack the cancerous cells in the bone marrow cell recipient. This process can also cause stem cells to be lost so that additional steps to prevent the loss of the stem cells are needed, for instance by using monoclonal antibodies that recognize the stem cells. Further, important cells called facilitator cells are lost. The loss of facilitator and stem cells increases the chances that the bone marrow cell graft will not succeed, i.e., will fail to engraft.

Another approach is to use a drug such as Cyclosporine (CSA). As previously discussed, CSA is an immunosuppressive drug that suppresses the function of the donor's T-cells. For patients not receiving a T-cell depleted transplant, the use of methotrexate added to Cyclosporine may be effective in decreasing the severity of GVHD. The side effects of Methotrexate include temporary but painful mouth sores that cause difficulty in eating and swallowing and reversible liver damage.

Chronic GVHD is the late form of GVHD. It may be caused by donated bone marrow T-cells which have grown up in the patient without maturing normally. The symptoms of chronic GVHD resemble many spontaneously occurring autoimmune disorders. Chronic GVHD occurs in about 40% of patients receiving an allogeneic transplant. Treatments include the use of Thalidomide and Cyclosporine. Chronic GVHD causes the death of about 10% of all allogeneic bone marrow cell recipients.

Tolerance by Establishment of Mixed Chimerism

Stable mixed chimerism can induce tolerance of transplanted organs and tissues. Various approaches have been used to achieve mixed chimerism. One approach has been to expose the recipient to high levels of radiation (called total body irradiation, TBI) and then infuse a mixture of donor and recipient bone marrow cells wherein the donor bone marrow cells have been treated to remove lymphocytes. (Sachs et al., Ann. Thorac. Surg., 56:1221 (1993); Illstad et al., Nature, 307:168 (1984)). Lower doses of TBI have also been used and followed by infusion of donor bone marrow cells plus antibodies against CD4 positive T-cells and CD8 positive T-cells and also natural killer cells to cause a general inhibition of immune function (Tomita et al., Transplantation, 61:469 (1996)). Others have used TBI plus a very high number of donor-derived hematopoietic cells that have been depleted of T-cells (Reisner et al., Immunol. Today 16:437 (1995); Bachar-Lustig et al., Nature Medicine, 12:1268 (1986)). TBI plus CY has also been reported.

Another approach is total lymphoid irradiation (TLI). In this approach, high doses of radiation (3,400-4,440 Gy) are used followed by infusion with donor bone marrow cells. TLI strongly suppresses the immune system. TLI reduces exposure of the recipient's bone marrow cell. This technique involves large amounts of radiation, repeated and lengthy in-clinic treatment, and has significant side effects.

Other variations of TLI and TBI treatments have been reported, for example, by Slavin and colleagues (PCT Publication No. WO 00/40701 A3, filed Dec. 23, 1999). Ildstad (U.S. Pat. No. 5,876,692) reports that anti-lymphocyte globulin (ALG) may be used to decrease the amount of TBI or TLI dosage. Other toleration protocols have been claimed, such as by Sachs in U.S. Pat. No. 5,876,708 wherein hematopoietic stem cells are introduced into a recipient, the recipient's T-cells are inactivated, the patient is immunosuppressed without recourse to antibodies against T-cells, and the recipient receives a graft from the donor. Other protocols claimed are, for instance, by Sykes in U.S. Pat. No. 6,006,752, which has claims to the creation of thymic space by irradiation or certain drug combinations.

One attempt to balance GVT with GVHD has been to infuse donor lymphocytes (DLI) into a recipient in incremental steps so as to provoke GVT and stop infusions after GVHD become too severe or difficult to control (Morecki and Slavin, J. Hematotherapy & Stem Cell Res 9:355, 357 (2000)). DLI has been performed before and after transplants but continues to carry significant risk of graft rejection or life-threatening GVHD. The need to balance GVT against GVHD is shown, for instance, in the attempt to promote GVT in a man that resulted in his death by GVHD (PCT Publication No. WO 00/40701 A3, Example 16).

Another attempted approach involves T-cell depletion, which is associated with a decrease in the risks of GVHD. Studies in rodents show that depleting T-cells can avoid GVHD risks (see Reich-Zeliger et al., Immunity 13:507-515, 2000). This procedure, however, is time-consuming, labor-intensive, requires multiple patient visits, and is often associated with the failure of bone marrow cells to engraft.

In animal models, it has been demonstrated that allogeneic bone marrow cell transplantation is a powerful treatment for various autoimmune diseases. However, the clinical application of bone marrow cell transplantation for nonmalignant diseases has been extremely limited, because these approaches largely rely on irradiation and treatments that severely suppress the immune and/or hematopoietic systems. These approaches are too toxic for widespread use in humans.

Bone marrow cell transplantation with such protocols induced either full chimerism or mixed chimerism in preconditioned hosts. In the setting of organ tissue transplants and autoimmune disease, low levels of stable donor mixed chimerism may be adequate to induce tolerance and continue autoreactivity. An early study by Cobbold et al., demonstrated that allogeneic bone marrow cell engraftment and specific tolerance could be achieved by a sublethal dose of total body irradiation and treatment of deleting anti-CD4 and anti-CD8 monoclonal antibodies. Subsequently, mixed chimerism as an approach for inducing tolerance in small animal models was extensively investigated using irradiation as a conditioning therapy. See Mixed Chimerism as an Approach for the Induction of Transplantation Tolerance, T. Wekerle and M. Sykes, Transplantation 68:459-467, 1999; and Mixed Chimerism as an Approach to Transplantation Tolerance, D. H. Sachs, Clinical Immunol. 95: S63-S68, 2000.

Recent studies report that mixed chimerism could also be induced by using costimulatory blockade and high-dose bone marrow cell transplantation (See Allogeneic Bone Marrow Transplantation With Co-Stimulatory Blockade Induces Macrochimerism and Tolerance Without Cytoreductive Host Treatment, T. Wekerle, J. Kurtz, H. Ito, J. V. Ronquillo, V. Dong, G. Zhao, J. Shaffer, M. H. Sayegh, and M. Sykes, Nat. Med. 6:464-469, 2000) or repeated bone marrow cell transplants. See Cutting Edge Administration of Anti-CD40 Ligand and Donor Bone Marrow Leads to Hemopoietic Chimerism and Donor-Specific Tolerance Without Cytoreductive Conditioning, M. M. Durham, A. W. Bingaman, A. B. Adams, J. Ha, S. Y. Waitze, T. C. Pearson, and C. P. Larsen, J. Immunol. 165:1-4, 2000. Hale et al., also reported that stable mixed chimerism can be established by a high dose of bone marrow cell, anti-lymphocyte serum (ALS), and rapamycin treatment. See Establishment of Stable Multilineage Hematopoietic Chimerism and Donor-Specific Tolerance Without Irradiation, D. A. Hale, R. Gottschalk, A. Umemura, T. Maki, and A. P. Monaco, Transplantation 69:1242-1251, 2000. However, these protocols are difficult to apply clinically because of the total amount of bone marrow cell required for transplantation. With a small amount of bone marrow cell, Tomita et al., showed that mixed chimerism could be induced in fully MHC-mismatched mice after donor spleen cell pretreatment followed myclosuppressive busulfan and cyclophosphamide. See Induction of Permanent Mixed Chimerism and Skin Allograft Tolerance Across Fully MHC-Mismatched Barriers by the Additional Myelosuppressive Treatment in Mice Primed With Allogeneic Spleen Cells Followed by Cyclophosphamide, Y. Tomita, M. Yoshikawa, Q. W. Zhang, I. Shimizu, S. Okano, T. Iwai, H. Yasui, and K. Nomoto, J. Immunol. 165:34-41, 2000.

Mixed Chimerism Established by the Present Invention

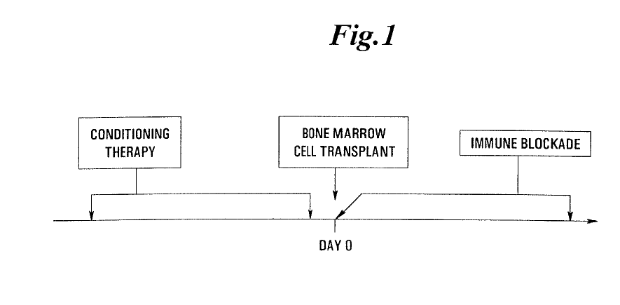

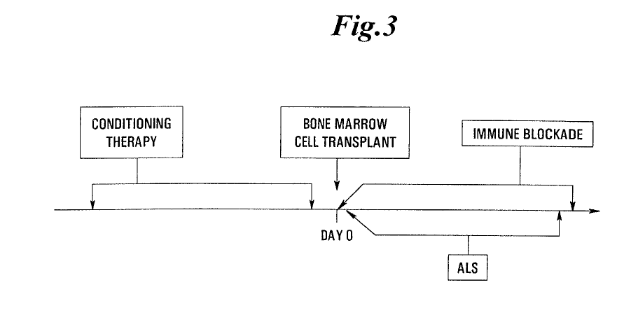

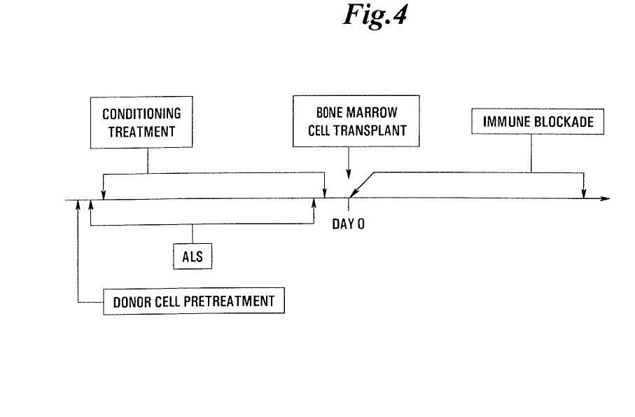

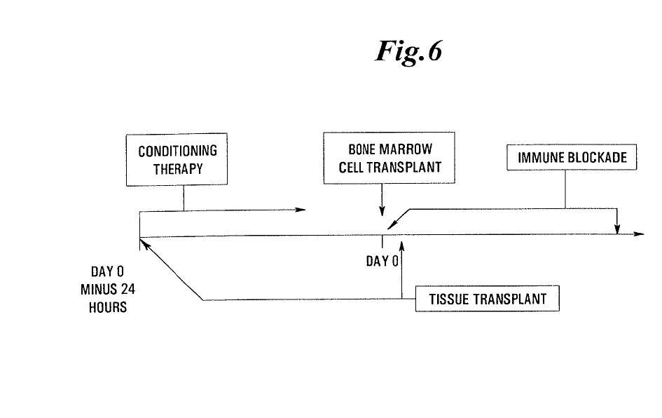

Referring to FIGS. 1-16, the preferred embodiments of the invention will be described. Chemically induced diabetic models have generally been used for islet transplantation and immune tolerance. However, they cannot truly reflect the clinical setting of autoimmune diabetes. It is for this reason that the NOD mouse has been extensively used as an animal model of human type 1 diabetes. The development of diabetes in these mice has been attributed to autoreactive T-cells that infiltrate pancreatic islets and specifically destroy insulin-producing islet beta cells. Islet allografts in diabetic NOD mice are destroyed by both alloimmune and recurrent T-cell-mediated anti-islet autoimmune responses (allograft means a graft from another individual of the same species; alloimmune means the immune system of another individual of the same species). The NOD mouse model is the best available model for experimental islet transplant research and predictive for the development of clinically relevant methods to induce and restore tolerance in humans.

A multitude of strategies have been shown to prevent the development of diabetes in NOD mice. Sublethal irradiation is one approach proven to prevent graft rejection and autoimmune destruction of islet allografts in overtly diabetic NOD mice. This approach establishes mixed allogeneic chimerism that simultaneously induces donor-specific tolerance to islet allografts and restores self-tolerance to islet autoantigens. See Allogeneic Chimerism Induces Donor-Specific Tolerance to Simultaneous Islet Allografts in Non-Obese Diabetic Mice, H. Li, C. L. Kaufman, and S. T. Ildstad, Surgery 118:192-197, 1995; and Allogeneic Hematopoietic Chimerism in Mice Treated With Sublethal Myeloablation and Anti-CD154 Antibody, Absence of Graft-versus-Host Disease, Induction of Skin Allograft Tolerance, and Prevention of Recurrent Autoimmunity in Islet-Allografted NOD/Lt Mice, E. Seung, N. Iwakoshi, B. A. Woda, T. G. Markees, J. P. Mordes, A. A. Rossini, and D. L. Greiner, Blood 95:2175-2182, 2000. Since NOD mice are irradiation-resistant, a high dose of irradiation is required to establish mixed chimerism, compared with other mouse strains. See Patterns of Hemopoietic Reconstitution in Non-Obese Diabetic Mice: Dichotomy of Allogeneic Resistance Versus Competitive Advantage of Disease-Resistant Marrow, C. L. Kaufman, H. Li, and S. T. Ildstad, J. Immunol. 158:2435-2442, 1997. Such high-doses of irradiation, however, are unacceptable for the establishment of mixed chimerism in patients with diabetes. Indeed, it has proved extremely difficult to prevent rejection, to prevent autoimmune destruction, and to induce tolerance in overtly diabetic NOD mouse recipients (Immunosuppression Preventing Concordant Xenogeneic Islet Graft Rejection is not Sufficient to Prevent Recurrence of Autoimmune Diabetes in Non-Obese Diabetic Mice, Z. Guo, D. Mital, J. Shen, A. S. Chong, Y. Tian, P. Foster, H. Sankary, L. McChesney, S. C. Jensik, and J. W. Williams, Transplantation 65:1310-1314, 1998). See NOD Mice Have a Generalized Defect in Their Response to Transplantation Tolerance Induction Diabetes, T. G. Markees, D. V. Serreze, N. E. Phillips, C. H. Sorli, E. J. Gordon, L. D. Shultz, R. J. Noelle, B. A. Woda, D. L. Greiner, J. P. Mordes, and A. A. Rossini, Diabetes 48:967-974, 1999, and Immunotherapy With Nondepleting Anti-CD4 Monoclonal Antibodies but not CD28 Antagonists Protect Islet Graft in Spontaneously Diabetic NOD Mice From Autoimmune Destruction, Allogeneic and Xenogeneic Rejection, Z. Guo, T. Wu, N. Kirchof, D. Mital, J. W. Williams, M. Azuma, D. E. R. Sutherland, and B. J. Hering, Transplantation, In press, 2001.

The preferred embodiment of the present invention includes a system of treatments for establishing mixed chimerism in mammals using a nonmyeloablative, nonirradiative approach. An optional treatment is donor cell pretreatment, which enhances the induction of mixed chimerism. Pretreatment by donor spleen cells is an example of donor cell pretreatment. The treatments are based on an appreciation of the function of the immune system and the function of medicinal tools that are used to control the immune system. The treatments, however, do not necessarily rely on any one particular theory of how the immune system or these medical tools function.

Mixed chimerism may be used to treat autoimmune diseases, including diabetes. Establishing mixed chimerism with the procedures of the invention prevents the onset of diabetes. Mixed chimerism probably favors migration of donor-derived cells to the recipient's thymus, where presentation of autoantigens by donor-derived antigen-presenting cells overcomes defective negative thymic selection of autoreactive T cells. As a result, autoreactive T cells undergo apoptosis in the thymus before appearing in the peripheral circulation. In addition, other mechanisms involving deletional and regulatory pathways are theorized to be involved in the restoration of self-tolerance.

Several observations and factors contribute to the system of treatments. Fludarabine phosphate (FL) is one of the purine nucleoside analogues that has immunosuppressive activity against lymphocytes in inhibiting DNA synthesis (See Metabolism and Action of Fludarabine Phosphate, W. Plunkett, P. Huang, and V. Gandhi, Semin. Oncol. 17:3-17, 1997) and by inducing apoptosis. See Differential Induction of Apoptosis by Fludarabine Monophosphate in Leukemic B and Normal T-Cells in Chronic Lymphocytic Leukemia, U. Consoli, I. El Tounsi, A. Sandoval, V. Snell, H. D. Kleine, W. Brown, J. R. Robinson, F. DiRaimondo, W. Plunkett, and M. Andreeff, Blood 91:1742-1748, 1998. CD4 and CD8 T cells are more sensitive to the effects of FL than B cells. See Fludarabine Phosphate: A DNA Synthesis Inhibitor With Potent Immunosuppressive Activity and Minimal Clinical Toxicity, E. R. Goodman, P. S. Fiedor, S. Fein, E. Athan, and M. A. Hardy, Am. Surg. 62:435-442, and Severe Immunodeficiency in Patients Treated With Fludarabine Monophosphate, P. W. Wijermans, W. B. Gerrits, and H. L. Haak, Eur. J. Haematol. 50:292-296, 1993. FL is therapeutically efficacious in the treatment of leukemia and lymphoma. See Fludarabine Phosphate: A New Active Agent in Hematologic Malignancies, M. J. Keating, S. O'Brien, W. Plunkett, L. E. Robertson, V. Gandhi, E. Esty, M. Dimopoulos, F. Cabanillas, A. Kemena, and H. Kantarjian, Semin. Hematol. 31:28-39, 1994. Since it induces lymphocytopenia, is highly immunosuppressive, and has mild nonhematologic toxicity; it has been successfully used as a nonmyeloablative conditioning regimen, combined with cyclophosphamide (CY) for human bone marrow cell transplantation. See Transplant-lite: Induction of Graft-versus-Malignancy Using Fludarabine-based Nonablative Chemotherapy and Allogeneic Blood Progenitor-Cell Transplantation as Treatment for Lymphoid Malignancies, I. F. Khouri, M. Keating, M. Korbling, D. Przepiorka, P. Anderlini, S. O'Brien, S. Giralt, C. Ippoliti, B. von Wolff, J. Gajewski, M. Donato, D. Claxton, N. Ueno, B. Andersson, A. Gee, and R. Champlin, J. Clin. Oncol. 16:2817-2824, 1998; and Low Intensity Regimens With Allogeneic Hematopoietic Stem Cell Transplantation as Treatment of Hematologic Neoplasia, A. M. Carella, S. Giralt, and S. Slavin, Haematologica, 85:304-313, 2000. CY, an alkylating agent, is immunosuppressive and is not marrow ablative in some situations. Spleen cell or bone marrow cell pretreatment of the host followed by CY administration induces microchimerism and donor-specific tolerance in most H-2 matched combinations, (See Drug-Induced Tolerance to Allografts in Mice IX. Establishment of Complete Chimerism by Allogeneic Spleen Cell Transplantation From Donors Made Tolerant to H-2-Identical Recipients, H. Mayumi, K. Himeno, K. Tanaka, N. Tokuda, J. L. Fan, and K. Nomoto, Transplantation, 42:417-422, 1986; and Intrahymic Clonal Deletion of V Beta 6⁺ T-Cells in Cyclophosphamide-Induced Tolerance to H-2-Compatible, Mls-Disparate Antigens, M. Eto, H. Mayumi, Y. Tomita, Y. Yoshikai, and K. Nomoto, J. Exp. Med. 171:97-113, 1990) but not in fully H-2 mismatched combinations. See Induction of Permanent Mixed Chimerism and Skin Allograft Tolerance Across Fully MHC-Mismatched Barriers by the Additional Myelosuppressive Treatment in Mice Primed With Allogeneic Spleen Cells Followed by Cyclophosphamide, Y. Tomita, M. Yoshikawa, Q. W. Zhang, I Shimizu, S. Okano, T. Iwai, H. Yasui, and K. Nomoto, J. Immunol. 165:34-41, 2000; and Evidence for Involvement of Clonal Anergy in MHC Class I and Class II Disparate Skin Allograft Tolerance After the Termination of Intrathymic Clonal Deletion, Y. Tomita, Y. Nishimura, N. Harada, M. Eto, K. Ayukawa, Y. Yoshikai, and K. Nomoto, J. Immunol. 145:4026-4036, 1990. FL and CY treatment eliminates lymphocytes in the host, but only slightly affects granulocytes and monocytes. These treatments with FL and CY, however, do not address GVHD, which is the major barrier to successful clinical bone marrow cell transplantation.

CD40/CD154 interaction is thought to be critical to induce both the humoral and the cellular immune response. See Immune Regulation by CD40 and Its Ligand GP39, T. M. Foy, A. Aruffo, J. Bajorath, J. E. Buhlmann, and R. J. Noelle, Annu. Rev. Immunol. 14:591-617, 1996; and CD40 and Its Ligand in Host Defense, R. J. Noelle, Immunity. 4:415-419, 1996. In the mouse model administration of anti-CD154 mAb alone, or in conjunction with donor cell treatment, prevented allogeneic heart, islet, and skin graft rejection (See Survival of Mouse Pancreatic Islet Allografts in Recipients Treated With Allogeneic Small Lymphocytes and Antibody to CD40 Ligand, D. C. Parker, D. L. Greiner, N. E. Phillips, M. C. Appel, A. W. Steele, F. H. Durie, R. J. Noelle, J. P. Mordes, and A. A. Rossini, Proc. Natl. Acad. Sci. U.S.A. 92:9560-9564, 1995; and Costimulatory Function and Expression of CD40 Ligand, CD80, and CD86 in Vascularized Murine Cardiac Allograft Rejection, W. W. Hancock, M. H. Sayegh, X. G. Zheng, R. Peach, P. S. Linsley, and L. A. Turka, Proc. Natl. Acad. Sci. U.S.A. 93:13967-13972, 1996) and induced tolerance. See CTLA4 Signals Are Required to Optimally Induce Allograft Tolerance With Combined Donor-Specific Transfusion and Anti-CD154 Monoclonal Antibody Treatment, X. X. Zheng, T. G. Markees, W W. Hancock, Y. Li, D. L. Greiner, X. C. Li, J. P. Mordes, M. H. Sayegh, A. A. Rossini, and T. B. Strom, J. Immunol. 162:4983-4990, 1999. However, it has been reported that CD154 is not an important costimulatory molecule of direct CD8+cell activation and CD40/CD154 independent activation of CD8+T cells can cause allograft rejection. See CD40-CD40 Ligand-Independent Activation of CD8⁺ T Cells Can Trigger Allograft Rejection, N. D. Jones, A. van Maurik, M. Hara, B. M. Spriewald, O. Witzke, P. J. Morris, and K. J. Wood, J. Immunol. 165:1111-1118, 2000. Tolerance to allografts induced anti-CD154 mAb and donor-specific transfusion is in part through deleting alloreactive CD8+T cells. See Treatment of Allograft Recipients With Donor-Specific Transfusion and Anti-CD154 Antibody Leads to Deletion of Alloreactive CD8⁺ T Cells and Prolonged Graft Survival in a CTLA4-Dependent Manner, N. N. Iwakoshi, J. P. Mordes, T. G. Markees, N. E. Phillips, A. A. Rossini, and D. L. Greiner, J. Immunol. 164:512-521, 2000. Anti-CD154 mAb blocked the development of acute and chronic GVHD. See Antibody to the Ligand of CD40, gp39, Blocks the Occurrence of the Acute and Chronic Forms of Graft-vs.-Host Disease, F. H. Durie, A. Aruffo, J. Ledbetter, K. M. Crassi, W. R. Green, L. D. Fast, and R. J. Noelle, J. Clin. Invest. 94:1333-1338, 1994; and Blockade of CD40 Ligand-CD40 Interaction ImpairsCD4⁺ T-Cell-Mediated Alloreactivity by Inhibiting Mature Donor T-Cell Expansion and Function After Bone Marrow Transplantation, B. R. Blazar, P. A. Taylor, A. Panoskaltsis-Mortari, J. Buhlmann, J. Xu, R. A. Flavell, R. Korngold, R. Noelle, and D. A. Vallera, J. Immunol. 158:29-39, 1997. The effect was attributed to the exhaustion of deletion of alloreactive DC8⁺-T-cell clones. See Cutting Edge: Sustained Expansion of CD8⁻ T-Cells Requires CD154 Expression by Th Cells in Acute Graft Versus Host Disease, J. E. Buhlman, M. Gonzalez, B. Ginther, A Panoskaltsis-Mortari, B. R. Blazar, D. L. Greiner, A. A. Rossini, R. Flavell, and R. J. Noelle, J. Immunol. 162:4373-4376, 1999. Blockade of CD40/CD154 interaction also prevented CD4⁺ T-cell mediated bone marrow cell graft rejection. Blockade of CD40 Ligand-CD40 Interaction ImpairsCD4⁺ T-Cell-Mediated Alloreactivity by Inhibiting Mature Donor T-cell Expansion and Function After Bone Marrow Transplantation, B. R. Blazar, P. A. Taylor, A. Panoskaltsis-Mortari, J. Buhlman, J. Xu, R. A. Flavell, R. Korngold, R. Noelle, and D. A. Vallera, J. Immunol. 158:29-39, 1997.

Another treatment is with Rapamycin. Rapamycin is a potent immunosuppressive agent. See Rapamune (Sirolimus, rapamycin). An Overview and Mechanism of Action, S. N. Sehgal, Ther. Drug Monit. 17:660-665, 1995. It has been used to prevent allograft rejection in humans. See Immunosuppressive Effects and Safety of a Sirolimus/Cyclosporine Combination Regimen for Renal Transplantation, B. D. Kahan, J. Podbielski, K. L. Napoli, S. M. Katz, H. U. Meier-Kriesche, and C. T. Van Buren, Transplantation 66:1040-10-46, 1998; and Sirolimus (Rapamycin)-Based Therapy in Human Renal Transplantation: Similar Efficacy and Different Toxicity Compared With Cyclosoprine. Sirolimus European Renal Transplant Study Group, C. G. Groth, L. Backman, J. M. Morales, R. Calne, H. Kreis, P. Lang, J. L. Touraine, K. Claesson, J. M. Campistol, D. Durand, L. Wrammer, C. Brattstrom, and B. Charpentier, Transplantation 67:1036-1042, 1999. Its mechanism of action is related to the blockade of signal transduction and inhibition of cell cycle progression. See Rapamune (RAPA, rapamycin, sirolimus): Mechanism of Action Immunosuppressive Effect Results From Blockade of Signal Transduction and Inhibition of Cell Cycle Progression, S. N. Sehgal, Clin. Biochem. 31:335-340, 1998. However, it has a primary effect on lymphokine responses rather than lymphokine production. In contrast to the calcineurin inhibitor, rapamycin does not block antigen priming activation-induced cell death. See Immunopharmacology of Rapamycin, RT. Abraham, and G. J. Wiederrrecht, Annu. Rev. Immunol. 14:483-510, 1996; and Two Distinct Signal Transmission Pathways in T Lymphocytes are Inhibited by Complexes Formed Between an Immunophilin and Either FK506 or Rapamycin, B. E. Bierer, P. S. Mattila, R. F. Standaert, L. A. Herzenberg, S. J. Burakoff, G. Crabtree, and S. L. Schreiber, Proc. Natl. Acad. Sci U.S.A. 87:9231-9235, 1990. Tolerance to allogeneic heart and skin grafts probably requires deletion of alloreactive T-cells through activation induced cell death. See Blocking Both Signal 1 and Signal 2 of T-Cell Activation Prevents Apoptosis of Alloreactive T-Cells and Induction of Peripheral Allograft Tolerance, Y. Li, X. C. Li, X. X. Zheng, A. D. Wells, L. A. Turka, and T. B. Strom, Nat. Med. 5:1298-1302, 1999; and Following the Fate of Individual T-Cells Throughout Activation and Clonal Expansion. Signals From T-Cell Receptor and CD28 Differentially Regulate the Induction and Duration of a Proliferative Response, A. D. Wells, H. Gudmundsdottir, and L. A. Turka, J. Clin. Invest. 100:3173-3183, 1997. Li et al., showed that rapamycin is very compatible with costimulation blockade. See Blocking Both Signal 1 and Signal 2 of T-Cell Activation Prevents Apoptosis of Alloreactive T-Cells and Induction of Peripheral Allograft Tolerance, Y. Li, X. C. Li, X. X. Zheng, A. D. Wells, L. A. Turka, and T. B. Strom, Nat. Med. 5:1298-1302, 1999; and Combined Costimulation Blockade Plus Rapamycin But Not Cyclosporine Produces Permanent Engraftment, Y. Li, X. X. Zheng, X. C. Li, M. S. Zand and, T. B. Strom, Transplantation 66:1387-1388, 1998. It has been suggested that anti-CD154 mAb alone cannot induce tolerance, which probably results from its inability to prevent graft rejection elicited by CD8+ T-cells. See CD40 Ligand Blockade Induces CD4+ T-Cell Tolerance and Linked Suppression, K. Honey, S. P. Cobbold, and H. Waldmann, J. Immunol. 163:4805-4810, 1999. Rapamycin was more effective in inhibiting CD8⁺ than CD4⁺-T-cell mediated GVHD. See Rapamycin Inhibits the Generation of Graft-versus-Host Disease—and Graft-versus-Leukemia-Causing T-Cells by Interfering With the Production of Th1 and Th1 Cytotoxic Cytokines, B. R. Blazar, P. A. Taylor, A Panoskaltsis-Mortari, and D. A. Vallera, J. Immunol. 160:5355-5365, 1998.

Induction of Mixed Chimerism

Mixed chimerism may be induced according to the present invention by performing a conditioning treatment, a bone marrow transplant, and an immune blockade (FIG. 1). The conditioning treatment mildly suppresses the immune system so that the transplanted bone marrow is not immediately rejected. The conditioning treatment avoids neutropenia and is only mildly myeloablative. The conditioning treatment prepares the recipient to receive the donor bone marrow. The bone marrow transplant involves taking bone marrow, stem cells, hematopoietic cells, immune system cells, or a combination of such cells from a donor and transplanting them into the recipient. Bone marrow transplantation may be performed in one medical procedure or in a series of smaller steps. Immune blockade prevents GVHD and enhances induction of mixed chimerism. It prevents the immune systems from attacking each other until they are fully integrated.

Conditioning Treatment

The conditioning treatment of the invention suppresses the recipient's immune system but avoids neutropenia and is nonmyeloablative or mildly myeloablative. In contrast, conventional conditioning treatments cause neutropenia and are not mildly myeloablative. Some current publications describe certain irradiation treatments as nonmyeloablative but such treatments are not nonmyeloablative in the sense that the invention is nonmyeloablative because the irradiation treatments destroy a large percentage of the patient's bone marrow cells and a substantially higher percentage than the treatments of the invention. In an alternate embodiment, other conditioning treatments that avoid neutropenia and are only mildly myeloablative may be used; for example, a regimen of irradiation administered at doses significantly less than practiced in conventional conditioning treatments.

The preferred embodiment of the invention uses FL and CY in combination for the conditioning therapy. Other combinations include busulfan alone or in combination with one or both of FL and CY. FL can be replaced by other purine nucleoside analogs, such as deoxycofornycin and 2-chloro-2′-deoxyadenosine and drugs with activity against dividing or non-dividing lymphocytes. CY may be replaced by other agents that may be used nonmyeloablatively such as ifosfamide, etoposide, mitoxantrone, doxorubicin, cisplatin, carboplatin, cytarabine, and paclitaxel. Low doses of drugs conventionally used or referred to as myeloablative drugs can be used in appropriate doses, such as nitrosoureas, melphalan, thiotepa, total body irradiation, and total lymphatic irradiation.

The conditioning treatment is preferably started and concluded when the bone marrow transplant is performed (FIG. 1). This timing is preferred because the immunosuppressive effect of the conditioning treatment prepares the recipient's immune system to cooperate with the donor immune system instead of attacking it. Thus, starting the conditioning treatment after the transplant is less preferred. The conditioning treatment may be started less than 48 hours before the bone marrow transplant. Preferably, the conditioning treatment is accomplished less than two weeks and optimally less than five days before the bone marrow transplant.

Bone Marrow Transplant

Bone marrow transplants may be performed in numerous ways known to those skilled in these arts. A common technique is to extract bone marrow from a donor's bones. The bone marrow may then be treated in a variety of ways; for example, the stem cells may be extracted and the bone marrow transplant accomplished by transplanting the stem cells to the recipient. Alternatively, stem cells may be recovered from a donor by other means, for example from their peripheral blood. The methods herein may be used with a human donor and also with a non-human, for example, a pig or primate.

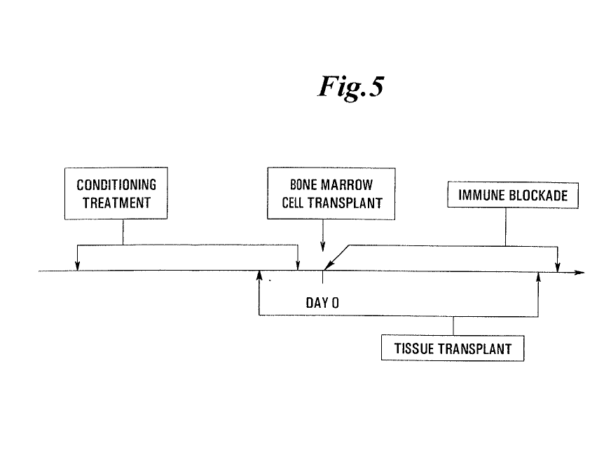

The bone marrow cell dosage and time of infusion may be varied, for example a modest dose of bone marrow may be infused several days before or after tissue transplantation (FIG. 5). The bone marrow transplant is preferably performed after the conditioning treatment has begun because it is desirable to at least mildly suppress the immune system to protect the transplanted cells. It is possible to overlap the beginning of bone marrow transplants with the end of conditioning therapy.

Immune System Blockade

The immune system blockade is preferably performed by use of agents that specifically suppress lymphocytes, preferably T-cells. Immune system blockade may include agents that block the T-cell co-stimulatory pathways, e.g., CTLA4Ig/LEA29Y or anti-CD154. Another preferred embodiment of the invention uses agents that block the response of T-cells to cytokines. e.g., rapamycin. Rapamycin may be replaced by immunosuppressants such as corticosteroids, methotrexate, cyclosporins, tacrolimus, mycophenolate mofetil, leflunomide, and FTY720.

The immune system blockade of the invention is used to prevent GVHD and to enhance chimerism. Since the blockade suppresses the activity of the donor cells it is preferable to begin the blockade at approximately the same time as the donor bone marrow is administered (FIG. 1). The use of immune blockade prior to transplant is possible but is inefficient.

Administration of Anti-Lymphocyte Serum (ALS)

The use of ALS is optional and is intended to enhance the induction of mixed chimerism ALS is specific to lymphocytes and suppresses the activity of host and donor immune systems. ALS is believed to enhance mixed chimerism by generally suppressing the immune systems and destroying clones of lymphocytes that react to the host or to the donor. Therefore, it is preferable to add ALS approximately when donor cells are introduced for the first time, either in the form of bone marrow cells or cells used for the cell pretreatment step. ALG, ATG, anti-CD3 mAb (OKT3), anti-CD4, and anti-CD8 are agents that may be used to replace ALS.

Rapamycin is preferably used in combination with the ALS treatment or its equivalent. The use of ALS and/or rapamycin may be replaced by costimulatory blockades such as anti-CD154 mAb, CTLA4Ig or anticytokine agents, for example anti-tumor necrosis factor, or regulatory cytokines, for example transforming growth factor beta or IL-10.

Donor Cell Pretreatment in Combination with ALS

Donor cell pretreatment is optional and may be used to enhance the induction of mixed chimerism. Donor cells are cells that display antigens to the recipient immune system that are given to the recipient prior to the bone marrow transplant. Spleen cells are useful donor cells but blood or cells taken from blood are also effective. The mechanism of the enhancement of chimerism is believed to be that the pretreatment cells trigger the recipient's immune system to begin to train lymphocytes and to amplify its response against the donor cells. Once this process is triggered, agents such as ALS may be added that partially destroy the recipient immune system's capability to respond to the donor cells. Donor cell pretreatment is preferably started prior to the infusion of bone marrow cells.

Donor Tissue Transplantation

Donor tissue transplants may be performed in numerous ways known to those skilled in these arts. The donated tissue is preferably transplanted 48 hours before or after the bone marrow transplantation so that tissue donation from a brain-dead organ donor (cadaveric donor) may readily be accomplished. A longer time period begins to introduce complications stemming from storage of the donor tissue. Alternatively, the bone marrow cell transplantation may be spread out into a number of doses over a time course or the donated tissue may be transplanted many days after the bone marrow cell transplantation.

The methods and systems of the present invention for producing mixed chimerism are effective for producing tolerance to any donated tissues. For example, tolerance may be induced that will allow safe transplantation of organs or tissues such as kidneys, livers, hearts, lungs, pancreas, small bowel, skin, neurons, and hepatocytes. Further, it is not necessary to limit transplantation to HLA-matched (MHC-matched) donors and recipients. Mismatches of more than 2 HLAs (2 MHC antigens) are possible.

### EXAMPLES

Materials and Methods

Many of the protocols and procedures are familiar to those skilled in these arts and are described in contemporary literature. The day of bone marrow cell transplantation is referred to as day 0, abbreviated d0; similarly 2 days before is d-2 and 2 days after is d2.

### Example 1

This example shows that donor cell pretreatment enhances the induction of allogeneic mixed hematopoietic chimerism in C57BL/6 and NOD mice when using nonirradiative and nonmyeloablative approaches. Allogeneic mixed hematopoietic chimerism can be used as an approach for inducting tolerance to alloantigens and restoring self-tolerance to autoantigens for islet transplantation. However, toxicity of conditioning therapy and the complication of bone marrow engraftment currently limits its clinical application. The NOD mouse strain, which is a mouse model of human type 1 diabetes, is irradiation-resistant and using conventional treatments, a high dose of irradiation has to be given in order to achieve mixed chimerism. The nonirradiative and nonmyeloablative fludarabine based conditioning therapies herein, however, produce sufficient immunosuppression to allow engraftment of allogeneic bone marrow cells. Anti-CD40 monoclonal antibody and rapamycin have been used to prevent the GVHD. This study showed that allogeneic mixed chimerism can be induced in C57BL/6 mouse strain and NOD mouse strain after transplantation of a modest bone marrow dose by using nonirradiative and nonmyeloablative fludarabine based approaches and that donor cell pretreatment enhances the induction of mixed chimerism. Balb/c spleen cells (H-2^{d}, 1×10⁸) were given intravenously (i.v.) at day-3 before bone marrow transplantation. Fludarabine (FL, 400 mg/kg) and cyclophosphamide (CY, 200 mg/kg) was given intraperitoneally (i.p.) at day-1. Each C57BL/6 mouse (H-2^{b}) or NOD mouse (H-2^{g7}) was infused with 4×10⁷ Balb/c bone marrow cells at day 0. Rapamycin (Rapa) was administrated by gavage at the dose of 2 mg/kg from day 0 to day 2, then 1 mg/kg once very two days until day 14. Anti-CD40L (MR1, 0.5 mg) was given i.p. at day 0 to day 5, then at day 7, 10 and 14. The level of donor-specific chimerism in peripheral blood was determined at different time points by flow cytometric analysis. Total number of chimeric mice and percentage of donor chimerism are shown as follows:

| | Donor | | | |
|---|---|---|---|---|
| Conditioning | Cell | Immune | Mixed Chimerism | |
| Therapy | Treatment | Blockade | 4 Weeks | 8 Weeks |

Induction of Mixed Chimerism in Balb/c to C57BL Strain Combination

| | | | | |
|---|---|---|---|---|
| FL + CY | No | Rapa | 4/5, | 4/5, |
| | | | 7.7 ± 1.0% | 10.3 ± 1.9% |
| FL + CY | No | MR1 | 6/6, | 5/5, |
| | | | 34.5 ± 20.9% | 28.5 ± 10.3% |
| FL + CY | No | MR1 + Rapa | 5/5, | 4/5, |
| | | | 9.0 ± 7.4% | 8.7 ± 4.6% |
| FL + CY | Yes | MR1 + Rapa | 6/6, | 6/6, |
| | | | 21.6 ± 4.3% | 24.9 ± 2.8% |
| FL | Yes | MR1 + Rapa | 0/6 | 0/6 |
| CY | Yes | MR1 + Rap | 5/6, | 5/6, |
| | | | 11.5 ± 1.7% | 14.3 ± 2.7% |

Induction of Mixed Chimerism in Balb/C to NOD Strain Combination

| | | | | |
|---|---|---|---|---|
| FL + CY | No | MR1 | 5/5, | 5/5, |
| | | | 81.6 ± 14.1% | 86.2 ± 16.2 |
| FL + CY | No | MR1 + Rapa | 6/6, | 6/6, |
| | | | 24.5 ± 10.0% | 26.1 ± 6.5% |
| FL + CY | Yes | MR1 + Rapa | 8/8, | 8/8, |
| | | | 56.3 ± 6.9% | 54.0 ± 15.1% |
| FL | Yes | MR1 + Rapa | 0/6 | 0/6 |
| CY | Yes | MR1 + Rap | 6/6, | 5/5, |
| | | | 27.5 ± 1.7% | 17.3 ± 3.7% |

These studies demonstrated that high level of allogeneic mixed chimerism could be induced in C57BL/6 and NOD mice after transplantation of a modest bone marrow dose by using fludarabine and cyclophosphamide as conditioning therapy. Donor cell pretreatment enhances the induction of mixed chimerism.

### Example 2

The conditioning therapy using FLU and CY was shown to avoid neutropenia. Five C57BL/6 mice were given FLU (400 mg/kg) and CY (200 mg/kg) as described in example 1 and five control mice received no treatment. After one week, blood samples were collecting and analyzed by flow cytometry using the CD3 marker for T cells and the CD45R/B220 marker for B cells. Lymphocytes (R1) in the treated mice were depleted by FL and CY treatment (FIGS. 8a and 8b) compared with the control mice (FIGS. 9a and 9b). But granulocytes (R2) and monocytes (R3) were only slightly affected, showing that neutropenia was avoided.

### Example 3

These protocols for inducing mixed chimerism were found to cause the recipients to remove the donor-reactive T-cells from their blood. Balb/C mice express antigens that are attacked by V-Beta 5.5⁺ and V-Beta 11⁺ TCR bearing T-lymphocytes and therefore normal balb/C mice do not have V-Beta5.5⁺ and V-Beta11⁺ T-lymphocytes. Therefore when balb/C bone marrow is transplanted into other mouse strains, it is desirable that the recipient mice do not have lymphocytes that express V-Beta5.5⁺ and V-Beta11⁺. C57BL/6 mice, however, normally do have V-Beta5.5⁺ and V-Beta11⁺ lymphocytes. Therefore a mixed chimer that successfully integrates the immune systems of both Balb/C and C57BL/6 mice should not have V-Beta5.5⁺ and V-Beta11⁺ lymphocytes.

The protocols described herein were used to induce mixed chimerism was in C57BL/6 mice using Balb/c donor bone marrow FIG. 10). V-Beta usage of TCR was studied 20 weeks after bone marrow transplantation. These experiments showed that that V-Beta5.5⁺ and V-Beta11⁺ lymphocytes were almost completely eliminated in theses chimeric mice at 20 weeks after bone marrow (as shown by measurements of CD4+ lymphocytes). Control lymphocytes were lymphocyte levels were unchanged (measured V-Beta 8⁺ CD4⁺ T-cells). These experiments show that these methods for inducing mixed chimerism result in deletion of donor-reactive T-cells.

### Example 4

The donor immune system T-cells of the mixed chimers developed by the procedures described herein did not attack the host. The frequency of donor specific cytokine (interferon-gamma, IL-2, IL-4, and IL-5) producing T-cells in mixed chimeric NOD mice was measured by enzyme-linked immunospot assay (ELISPOT) assay a 20 weeks after bone marrow cell transplantation. Spleen cells from recipient chimeric mice and recipient non-chimeric mice were collected and cultured with donor cells or phytohemagglutinin (PHA) for 24 hours. Few donor specific cytokine producing T cells could be found in chimeric NOD mice compared to NOD mice without chimerism (FIG. 11). PHA mitogen specific cytokine secreting T cells were seen in both chimeric and non-chimeric NOD mice (FIG. 12).

### Example 5

The onset of diabetes in prediabetic mice was prevented by establishing mixed chimerism using the procedures described herein. NOD prediabetic mice were treated with conditioning treatment, bone marrow cell transplants, and immune blockade at 8-9 weeks of age and compared to untreated prediabetic NOD mice. Blood glucose levels were monitored (FIG. 13). At age 24 weeks, none of the 27 chimeric mice had developed diabetes but 61 of 100 of the control mice had developed diabetes. (p<0.01).

### Example 6

Diabetes was cured by inducing mixed chimerism in combination with a pancreatic islet transplant. NOD mice that had been diabetic for ate least two weeks were given a donor-cell pretreatment of Balb/c spleen cells (1×10⁸) at d-3. Fl (400 mg/kg) and CY (200 mg/kg) were given intraperitoneally on d-1. Balb/c bone marrow cells (4×10⁷) were given on d0. Rapamycin was administered by gavage (2 mg/kg/day) from d0 to d2 and then every other day at 1 mg/kg/day until d14. Anti-CD154 (MR1, 0.5 mg) was given intraperitoneally daily from d0 to d5, then on d7, d10, and d14. Flow cytometry was used to measure donor-specific chimerism two weeks after bone marrow cell transplant. All pancreatic islet grafts survived over 60 days in chimeric mice with mixed chimerism levels of at least 30% donor cells at two weeks (FIG. 14). Islet grafts were rejected in 5 of 7 chimeric mice with less than 30% donor chimerism.

### Example 7

Diabetes was cured by simultaneous bone marrow cell and pancreatic islets. Preconditioning treatments of FL (200 mg/kg) and CY (100 mg/kg) were administered intraperitoneally to female recipient NOD mice at d-2 and d-1. Anti-lymphocyte serum (ALS, 0.3 ml) was given on d-1 and on d0. Four hundred MHC-matched male NOR islets were transplanted into the left kidney capsule of each diabetic female NOD mouse, and 1×10⁸ male NOR bone marrow cells were simultaneously injected intravenously. Rapamycin was administered at 1/mg/kg from d0 to d2 and then very other day until d14. NOR islet survival without any treatment was 8.0±2 days. FL and CY treatment prolonged islet graft survival to 23.5±8.5 days (p<0.05). ALS and rapamycin treatment and NOR bone marrow cell infusion also significantly prolonged NOR islet graft survival to 32±2.5 days (p<0.01). However, all NOR islet grafts that survived over 100 days had simultaneous bone marrow cell/islet transplant and received Fl, CY, ALS, and rapamycin (Table Ex7-1). The return of hypoglycemia after nephrectomy confirmed that the islet grafts were functioning.

To further test whether donor-specific tolerance had been induced, donor NOR islets or third-party Balb/c islets were transplanted into the right kidney capsule of these mice. Donor-specific NOR islet grafts survived over 80 days and third-party Balb/c islet grafts were rejected in two weeks (Table Ex7-2). Donor-specific chimerism of peripheral blood in these mice was measured by semi-quantitative PCR for a male specific marker (SRY). The average percentage of this male NOR marker in DNA derived from peripheral blood of these female NOD mice at 100 days post-transplantation was 10%.

TABLE Ex-2

| Bone marrow cell Transplant | Conditioning Treatment | Islet Graft Survival (Days × n) |
|---|---|---|
| No | None | 5, 6 × 2, 7 × 2, 8 × 3, 9, 11 × 2, 12 |
| No | FL + Cy | 17, 23, 24, 40 |
| Yes | ALS + Rapamycin | 28, 32 × 2, 35 |
| Yes | FL + CY + ALS + Rapamycin | >100 × 7 |

TABLE Ex7-2
Second Islet Graft Survival In Diabetic NOD Mice

| Donor | Treatment | Islet Graft Survival (Days*) |
|---|---|---|
| Balb/c | None | 12, 14 |
| NOR | None | >100 × 4 |
| | | >60 × 3 |

*the symbol “>” indicates that the mice are still alive and have not rejected their graft at the time of writing

### Example 8

This example shows methods and systems for inducing mixed hematopoietic chimerism without irradiation in a fully MHC-mismatched allogeneic bone marrow transplantation. This example shows that stable and high levels of mixed chimerism can be induced by irradiation-free nonmyeloablative approaches after transplantation of regular does of bone marrow in a fully MHC-mismatched mouse combination. Donor-specific transfusion (DST, 0.25 ml) was given a day-7. ALS (0.3 ml) was administered at day-8 and day-5. Busulfan (Bu, 20 mg/kg) and cyclophosphamide (Cy, 100 mg/kg) was given at day-3 and day-2. Bone marrow at a dose of 4×10⁷ from Balb/c mice were injected into each C57BL/6 mice at day 0. Anti-CD40L (MR1, 0.5 mg) was give at day 0, 2 and CTLA4Ig was given at day 2. Rapamycin (Rapa) was administrated at the dose of 2 mg/kg from day-1 and day 2, then 1 mg/kg once very two days until day 14. The level of donor-specific chimerism was determined at different time points by flow cytometry. The results of different groups were as follows:

TABLE Ex8-1
Balb/c Donor Chimerism in PBL of C57BL/6 Mice at 8 Weeks Post-Transplant

| | | | | | Percentage of |
|---|---|---|---|---|---|
| | Conditioning | | | | Donor Cells |
| Group | Therapy | DST | Immune Blockade | Chimeric Mice | in Chimeric Mice |
| 1 | Bu + CY, ALS | No | MR1 + CTLA4Ig + Rapa | 5/5 | 34.3 ± 7.4% |
| 2 | Bu + CY, ALS | Yes | MR1 + CTLA4Ig + Rapa | 5/5 | 74.8 ± 4.8% |
| 3 | ALS | Yes | MR1 + CTLA4Ig + Rapa | 0/6 | 0% |
| 4 | Bu + CY | Yes | MR1 + CTLA4Ig + Rapa | 1/6 | 38.9% |
| 5 | Bu + CY, ALS | Yes | Rapa | 6/6 | 76.8 ± 13.6% |
| 6 | Bu + CY, ALS | Yes | MR1 + CTLA4Ig | 4/5 | 63.7 ± 7.0% |
| 7 | Bu + CY, ALS | Yes | MR1 | 4/6 | 25.3 ± 3.3% |
| 8 | Bu + CY, ALS | Yes | CTLA4Ig | 3/6 | 18.2 ± 12.9% |
| 9 | Bu + CY, ALS | Yes | MR1 + Rapa | 6/6 | 50.3 ± 4.0% |

FIG. 16 shows the donor chimerism levels at 20 weeks in various hematopoietic organs.

These studies demonstrated that stable and high level of mixed chimerism could be induced in a fully MHC-mismatched mouse combination after transplantation of regular dose of bone marrow without any irradiation. Bu+Cy and ALS as conditioning therapy successfully induced mixed chimerism. Costimulatory blockades and Rapamycin alone or combination as post-bone marrow treatment helped to induce mixed chimerism. This approach may be used to induce donor-specific tolerance in clinical islet transplantation and living donor related solid organ transplantation.

### Further Embodiments

A method of transplanting a donor tissue by administering a bone marrow cell transplant from a donor to a recipient; administering a conditioning treatment to the recipient that avoids neutropenia; administering an immune blockade treatment to the recipient, and transplanting a donor tissue from the donor to the recipient, wherein the donor is a clinical cadaver and the tissue transplant, conditioning treatment, and bone marrow cell transplant are all completed within a single continuous forty-eight hour period of time, or, more preferably, simultaneously. Further, such treatment may be controlled so that the conditioning treatment causes the amount of granulocytes in the recipient's blood to decrease by less than 30%. Also, the bone marrow cell transplant may be performed after the donor tissue transplant. The bone marrow cell transplant may be made by administering donor stem cells to the recipient, including stem cells collected from the donor's blood.

Typically, the donor's bone marrow cells are removed from the donor prior to inducing mixed chimerism in a patient; alternatively, a patient who will be treated may donate bone marrow that is transplanted into another person who will become a mixed chimer that will donate the mixed chimerism back to the patient. Thus a cancer patient may donate to an animal that will generate immunity against a cancer for the cancer patient; the animal may be a human or another mammal.

The conditioning treatment preferably uses a combination of fludarabine phosphate, busulfan, cyclophosphamide, and/or their equivalents. Agents for conditioning may include a purine nucleoside analog. The conditioning treatment may also use deoxycoformycin or 2-chloro-2′ deoxyadenosine or a drug chosen from the group consisting of ifosamide, etoposide, mitoxantrone, doxorubicin, cisplatin, carboplatin, cytarabine, and paclitaxel. The conditioning treatment may include a nitrosoureas, melphalan, or thiotepa.

The invention includes a convenient kit for inducing mixed chimerism so that clinicians, including non-doctors and nurses, may readily and confidently apply the invention. The kit may include conditioning treatment drugs, immune blockade drugs, and instructions for delivering the drugs in a sequence and at predetermined levels. Conditioning drugs may include fludarabine phosphate, busulfan, cyclophosphamide, purine nucleoside analogs, deoxycoformycin, 2-chloro-2′ deoxyadenosine, ifosamide, etoposide, mitoxantrone, doxorubicin, cisplatin, carboplatin, cytarabine, paclitaxel, nitrosoureas, melphalan, or thiotepa. The immune blockade drugs may include rapamycin. They may also be drugs that inhibit T-cell CD28 binding to B7 receptors.

The invention includes methods of inducing mixed chimerism in a bone marrow cell transplant recipient by administering a conditioning treatment to a recipient that avoids neutropenia; administering a bone marrow cell transplant from a donor to the recipient; and administering an immune blockade treatment to the recipient that causes lymphocyte-specific immune suppression; thereby causing the patient to express detectable mixed chimerism. The conditioning and transplant may be done within four weeks of each other although one week is more preferable and a simultaneous transplant is most preferable. Chimerism may be measured from samples of peripheral blood. Preferably at least 1% mixed chimerism is induced for most applications. Preferably at least 10% mixed chimerism is induced when treating autoimmune diseases.

Anti-lymphocyte serum (ALS) may be used as part of the methods of inducing mixed chimerism and typically enhanced the induction of mixed chimerism. ALS may be administered, for example, within 48 hours after the end of the donor cell pretreatment or, for example, 48 hours after the bone marrow transplant.

The invention includes a method of transplanting cells from a donor into a recipient that causes the transplanted cells to contribute to the function of the donor's immune system, the method having a step of preparing the recipient with a conditioning treatment that reduces the number of neutrophil cells by no more than 30%; a step of transplanting immune system cells from the donor into the recipient; and a step of immune blockade.

Tissue donors may be living or cadaveric, for example, a living or cadaveric pancreatic islet or kidney donor.

The invention also includes a method of transplanting pancreatic islet cells from a donor to a recipient by administering a bone marrow cell transplant and a pancreatic islet cell transplant from a donor to a recipient within a 96 hour time period; administering a conditioning treatment to the recipient that is mildly myeloablative, and administering an immune blockade treatment to the recipient. The mildly myeloablative treatment may be performed with fludarabine phosphate or cyclophosphamide. Moreover, the bone marrow cell transplant and pancreatic islet cell transplant could performed within a twelve hour time period or even, preferably, simultaneously. The method may be administered so that it causes a donor chimerism level of at least 30% as determined by measurements taken from peripheral blood samples.

The invention includes animals that are mixed chimers and mixed chimers made by these processes. It includes, for example, a medically modified animal having a mixed chimerism immune system created by the process of administering a bone marrow cell transplant from a donor to an animal; administering a mildly myeloablative conditioning treatment to the animal, and administering an immune blockade treatment to the animal. The animal includes mice, pigs, and monkeys. The donor may be an animal or a human.

The invention includes a method of transplanting a donor tissue by administering a bone marrow cell transplant from a donor to a recipient; administering a nonmyeloablative conditioning treatment to the recipient, administering an immune blockade treatment to the recipient, and transplanting a donor tissue from the donor to the recipient, wherein the donor is a non-human. The donor tissue may include cells from a pancreatic islet.

The systems and methods of the invention include cancer treatments. The immune system normally removes cells that have transformed into potentially cancerous cells but the immune system sometimes fails to recognize the transformed cells with the result that they multiply and spread through the body, a situation generally termed cancer. Since a person who is a mixed chimer effectively uses both their original immune system and the donated immune system, the donated immune system is able to attack the patient's cancer. Indeed, modem bone marrow cancer treatment by removal of all of the bone marrow followed by engraftment of donor bone marrow is directed not to removing every single cancerous bone marrow cell but towards establishing full chimerism. The present invention uses mixed chimerism to treat cancer. For example, a cancer patient may be the recipient of a bone marrow transplant and made into a mixed chimer. Inducing mixed chimerism may activate the GVT effect so that the cancer is treated.

The systems and methods of the invention include treatments for autoimmune diseases. Induction of mixed chimerism may be performed to retrain the recipient's immune system to recognize the “self” properly. Further, mixed chimerism may be used to prevent the onset of autoimmune disease or cancer. For example, patients that are known to be at risk for diabetes or certain cancers may be made into chimers so that they do not develop cancer or diabetes.

1. A method of transplanting a donor tissue, the method comprising the steps of administering a bone marrow cell transplant from a donor to a recipient; administering a conditioning treatment to the recipient that avoids neutropenia; administering an immune blockade treatment to the recipient, and transplanting a donor tissue from the donor to the recipient, wherein the donor is a clinical cadaver and the tissue transplant, conditioning treatment, and bone marrow cell transplant are completed within a single continuous forty-eight hour period of time.

2. The inventions as described herein.
